(19) United States
(12) Patent Application Publication
(10) Pub. No.: US 2026/0088187 A1
(43) Pub. Date: Mar. 26, 2026

(54) METHOD AND DEVICE FOR CONVERTING CHEST RADIOLOGY DATA INTO NUMERICAL VECTOR, AND METHOD AND DEVICE FOR ANALYZING DISEASE BY USING SAME

(71) Applicant: SEOUL NATIONAL UNIVERSITY HOSPITAL, Seoul (KR)

(72) Inventor: Joonghee KIM, Seongnam-si, Gyeonggi-do (KR)

(21) Appl. No.: 18/872,245

(22) PCT Filed: Jun. 7, 2023
(86) PCT No.: PCT/KR2023/007755

§ 371 Date: Oct. 8, 2025

(30) Foreign Application Priority Data
Jun. 7, 2022 (KR) 10-2022-0069096
May 12, 2023 (KR) 10-2023-0061865

(51) Int. Cl. G16H 70/60

(52) U.S. Cl. CPC G16H 70/60

(57) ABSTRACT

Exemplary implementations of the present application include a device for analyzing a disease by converting chest radiology data into numerical vectors, the device comprising: an acquisition unit for acquiring chest radiology data; an encoder, that receives the chest radiology data and uses a deep learning algorithm so as to calculate a first numerical vector; and an analysis unit, that uses the first numerical vector calculated by the encoder, so as to provide an analysis result that is information regarding disease-related analysis, prediction, or diagnosis, wherein the first numerical vector is structured data contextually including anatomical features that can be extracted from the chest radiology data, and being associated with features extracted from the chest radiology data.

### TECHNICAL FIELD

This specification relates to a method and a device for converting a chest radiology image into numerical vectors, and to a method and a device for analyzing and predicting a disease, and providing diagnostic assistance information related to the disease using the same.

### CROSS-REFERENCE TO RELATED APPLICATIONS

The present application claims priority to Korean Patent Application No. 10-2022-0069096, filed on Jun. 7, 2022, and Korean Patent Application No, 10-2023-0061865, filed on May 12, 2023, the entire contents of which are incorporated herein by reference.

### BACKGROUND ART

Chest radiology is a very commonly used test in clinical practice. In chest radiology images, various organs, such as lungs, heart, aorta, ribs, sternum, and vertebrae, can be observed. Thus, the chest radiology images can be used to diagnose a wide variety of anatomical variations and diseases. In recent years, various artificial intelligence systems for analyzing chest radiology images have been developed, but these artificial intelligence systems have been optimized to perform only in limited areas where they were developed. The artificial intelligence systems also have had several limitations in analyzing with high accuracy various diseases that can be theoretically covered by chest radiology, and temporal changes thereof.

### PRIOR ART DOCUMENT

### Patent Document

(Patent Document 1) Korean Patent Registration Publication No. 10-2163217 (Oct. 8, 2020)

### DETAILED DESCRIPTION OF THE INVENTION

### Technical Solution

In one aspect, in exemplary implementations of the present application, provided are a method and a device for analyzing and predicting a disease, or providing diagnostic assistance information by extracting numerical information to maximize the utilization range of chest radiology data, utilizing the same within a clinical framework, or fusing the same with other information of a patient.

### SUMMARY OF THE INVENTION

In exemplary implementations of the present application, there is provided a device for analyzing a disease by converting chest radiology data into numerical vectors, the device including: an acquisition unit for acquiring chest radiology data; an encoder that receives the chest radiology data and uses a deep learning algorithm so as to calculate a first numerical vector; and an analysis unit that uses the first numerical vector calculated by the encoder so as to provide an analysis result that is information regarding disease-related analysis, prediction, or diagnosis, wherein the first numerical vector is structured data contextually including anatomical features that can be extracted from chest radiology data, and being associated with features extracted from the chest radiology data.

In addition, in other exemplary implementations of the present application, there is provided one or more downstream task processing units that utilize the first numerical vector to process a downstream task.

In addition, in other exemplary implementations of the present application, there is provided a method of analyzing a disease by converting chest radiology data into numerical vectors, performed by a processor, the method including: obtaining chest radiology data from a chest radiology measurement device; inputting the chest radiology data into an encoder; calculating a numerical vector using deep learning through the encoder; performing disease-related analysis, prediction, or diagnosis using the numerical vector; and processing one or more downstream tasks utilizing the numerical vector.

Specifically, in one aspect, the first numerical vector is structured data associated with features extracted from chest radiology data including, particularly contextually including, anatomical (positional) features that can be extracted from the chest radiology data. This first numerical vector is effectively used for the downstream task or machine learning as described below.

In an exemplary implementation of the device, error signals from an output end of a network of the downstream task of the one or more downstream task processing units may be backpropagated to gather at one encoder end to train one encoder, thereby improving the versatility of the first numerical vector.

In an exemplary implementation of the device, the first numerical vector may be used as an input vector of the downstream task processing unit by itself or may be concatenated with other structured data information.

In an exemplary implementation of the device, the number of encoders may be two or more, and a plurality of first numerical vectors output from each encoder may be concatenated to create one input numerical vector.

In an exemplary implementation of the device, N sequential chest radiology data may be passed through one encoder to obtain N sequential first numerical vectors.

In an exemplary implementation of the device, the device may be configured to fix weights of a network of the encoder when training the network of the downstream task, modify (update) the weights of the network of the downstream task through training, and then modify (update) all the weights of the networks of the encoder and the downstream task through additional training.

In an exemplary implementation of the device, each of the one or more downstream task processing units may be performed by a multi-layer perceptron (MLP) having two or more fully connected layers.

In an exemplary implementation of the device, the MLP may be trained through multi-task learning jointly with encoding network training of the encoder or may be separately trained after the encoder first completes the training.

In an exemplary implementation of the device, the MLP may receive additional structured data input information which is different from the first numerical vector, wherein the additional structured data input information corresponds to at least one or more of an age, a gender, vital signs (e.g., blood pressure, pulse, respiratory rate, body temperature, SpO2, and blood sugar), biosignals (e.g., electrocardiogram (ECG), photoplethysmography (PPG), electroencephalogram (EEG), and invasive pressure measurements of arteries and central veins), specimen test results (various blood tests, and biopsy), natural language information, and numerical or categorical data extracted from image data other than chest radiology data. The additional structured data input information may be concatenated with the first numerical vector or may be input separately from the first numerical vector.

In an exemplary implementation of the device, the device may further include a display unit configured to present a marginal probability of a specific disease considering chest radiology data obtained while outputting the MLP and a marginal probability of a specific disease without considering the obtained chest radiology data together as a baseline risk probability, when the MLP predicts the occurrence or non-occurrence of a specific disease, and may display how many times the marginal probability of a specific disease considering the obtained chest radiology data is increased in proportion to the marginal probability of a specific disease without considering the obtained chest radiology data.

In an exemplary implementation of the device, the deep learning algorithm of the encoder corresponds to a vision network based on the structure of a convolutional neural network (CNN) or a transformer (visual transformer: ViT). The structures of CNN and ViT may correspond to network structures commonly used for image data classification, and the classification performance and the efficiency thereof may be expanded through various modifications and extensions. In implementing the present application, selecting the CNN or the ViT of a specific structure belongs to a process optimized by the type and the amount of training data and the task being processed, and the encoder is not limited to the specific structure of the vision network, such as the CNN or the ViT.

In an exemplary implementation of the device, a subunit of the encoder includes: one or more convolution layers; one or more fully connected layers, wherein the fully connected layers include a non-linear activation function; and an attention layer that summarizes a feature set extracted from channel-specific chest radiology data to each extract a representative value and recalibrates the channel-specific feature set to reflect a contribution of the channel-specific feature set based on the representative value, wherein the feature set includes a morphological feature for each channel, and the recalibrated channel-specific feature set may have more focused morphological features for each channel than the feature set.

In an exemplary implementation of the device, the one or more convolution layers may include a depthwise-separable convolution layer that individually convolutes chest radiology data for each of the one or more channels.

In an exemplary implementation of the device, the attention layer may be configured to pool the feature set so as to summarize the feature set.

In an exemplary implementation of the device, the attention layer may be configured to pass a channel-specific representative value through the fully connected layer to calculate a channel-specific contribution, and multiply the channel-specific contribution by the feature set to recalibrate the channel-specific feature set.

In an exemplary implementation of the device, the attention layer may be configured to calculate the channel-specific contribution by scaling a result of passing the channel-specific representative value through the fully connected layer into a numerical value within a specific range.

In an exemplary implementation of the device, the encoder subunit includes a squeeze-excitation layer that extracts an average for each channel to calculate a scalar value, wherein the scalar value for each channel is between 0 and 1. The encoder subunit is scaled according to the importance of the channel, passes a vector in which the scalar value for each channel is gathered through the fully connected layer, and then applies a sigmoid/RELU function to reduce the dimension.

In an exemplary implementation of the device, the encoder may include a plurality of convolution blocks, and the subunit may be included in other convolution blocks than the first convolution layer.

In an exemplary implementation of the device, the convolution block includes a first encoder subunit and a second encoder subunit, wherein the first encoder subunit is applied closer to an input end than an output end of the convolution block, compared to the second encoder subunit. The attention layer may more focus on the operation of extracting a representative value by summarizing the feature set, than the second encoder subunit, among the operation of extracting a representative value by summarizing the feature set and the operation of recalibrating the feature set according to the channel-specific contribution, wherein the representative value of the first encoder subunit reflects the morphological features more than the representative value of the second encoder subunit. The second encoder subunit is applied closer to the output end than the input end of the convolution block, compared to the first encoder subunit. The attention layer may more focus on the operation of recalibrating the feature set according to the channel-specific contribution than the first encoder subunit among the operation of extracting the representative value by summarizing the feature set and the operation of recalibrating the feature set according to the channel-specific contribution.

In an exemplary implementation of the device, the last convolution block of the encoder may further include a non-local network, wherein the non-local network may be configured to compare the similarity between spatial points of the chest radiology data to implement spatial attention.

In an exemplary implementation of the device, the chest radiology data is a single-channel or multi-channel image. The chest radiology image data input to the encoder may be in the form of a two-dimensional or three-dimensional array of C×W×H (the number of channels x the number of horizontal pixels x the number of vertical pixels).

In an exemplary implementation of the device, the chest radiology data may be a chest radiology image, wherein the chest radiology image may be resized and cropped to a particular size and normalized to be input to the encoder.

In an exemplary implementation of the device, information about disease diagnosis provided by the analysis unit may include rhythm abnormalities of the heart, including at least one or more of tachycardia, bradycardia, and various arrhythmias, and structure and function abnormalities of the heart including at least one or more of heart failure, pericardial tamponade, valvular stenosis/failure, pulmonary hypertension, pulmonary embolism, and cardiomyopathy.

In an exemplary implementation of the device, the disease predicted and diagnosed by the analysis unit may include acute respiratory distress syndrome (ARDS), pneumonia, abscess, aspiration pneumonia, atypical pneumonia, active tuberculosis, non-tuberculous mycobacteria, chronic obstructive pulmonary disease (COPD), interstitial lung disease, bronchiectasis, sarcoidosis, lung nodule, lung mass, lung cancer, lung metastasis, aortic dissection, aortic aneurysm, pleural effusion, empyema, pneumothorax, pneumoperitoneum, pneumopericardium, pneumomediastinum, subcutaneous emphysema, coronary artery calcification, cardiomegaly, pulmonary edema, pericardial effusion, pulmonary embolism, chamber (LA, LV, RA, RV) enlargements, valvular (aortic, mitral, tricuspid, pulmonic) calcification/stenosis/regurgitation, hypertrophic cardiomyopathy, and various fractures, tumors, and metastasis of ribs, sternum, and spine.

In an exemplary implementation of the device, the analysis result may include disease diagnosis assistance information for determining whether the disease improved or worsened using the first numerical vector. When the analysis unit provides the disease diagnosis assistance information, the chest radiology data may be a plurality of pieces of chest radiology data measured at regular intervals. Each of the plurality of pieces of chest radiology data may pass through a pooling layer of the encoder to provide the diagnosis assistance information on whether the disease improved or worsened from the obtained first numerical vector.

In an exemplary implementation of the device, the analysis result may include providing the disease diagnosis assistance information. The chest radiology data is a plurality of pieces of chest radiology data measured at regular or irregular time intervals. The analysis unit may arrange each of the first numerical vectors of the plurality of pieces of chest radiology data as a sequential vector, may concatenate the sequential vectors in a vector length direction to pass through an MLP network, may concatenate the sequential vectors in a vertical direction to the vector length direction to pass through a transformer network, or may not concatenate the sequential vectors to sequentially pass through the RNN, and encode information about time using a function to extract a second numerical vector to diagnose the occurrence, improvement, or worsening of a patient's specific disease over time.

In an exemplary implementation of the device, the encoder may be trained through self-supervised learning based on clinically defined morphological features of chest radiology data.

In an exemplary implementation of the device, the encoder may be trained through self-supervised learning by using data obtained by modifying the chest radiology data in a certain manner as training data.

In an exemplary implementation of the device, when the original chest radiology data and the augmented data obtained by subjecting the original chest radiology data to data augmentation are input to the encoder, the encoder may include a process of training the encoder so that each of the calculated first numerical vectors is the same or highly similar.

In an exemplary implementation of the device, the process of calibrating each of the calculated first numerical vectors to be the same or highly similar may minimize the distance of each of the calculated first numerical vectors.

In an exemplary implementation of the device, the device may be medical equipment equipped with a chest radiology measurement device, a device equipped with a smartphone app, and an augmented reality device (a combination of a camera and glasses), or may be combined with an electronic health record system. In addition, the device may be implemented as an API system rather than specific equipment or software as described above. In this case, the device may also be implemented as a service (device) through which another equipment or system sends chest radiology data and sends an analysis result thereof back to the equipment or system.

On the other hand, in another aspect, a method of converting chest radiology data into numerical vectors, performed by a processor, or a method of analyzing a disease from chest radiology data, performed by a processor, using deep learning comprises: obtaining chest radiology data from the chest radiology measurement device; inputting the chest radiology data into the encoder; and calculating the first numerical vector using the deep learning algorithm through the encoder, wherein the first numerical vector may be structured data associated with features extracted from the chest radiology data including, particularly contextually including, anatomical (positional), physiological (functional), or pathological features that can be extracted from the chest radiology data. This first numerical vector is effectively used for downstream task or machine learning as described below.

In an exemplary implementation, the method may further include analyzing and predicting a disease or health, or providing diagnostic assistance information using the first numerical vector.

In an exemplary implementation, the method may include simultaneously processing a plurality of downstream tasks utilizing the first numerical vector. When error signals from each output end of the network of the downstream task are backpropagated, the error signals are gathered at one encoder end to train one encoder, thereby improving the versatility of the first numerical vector.

In an exemplary implementation of the method, the first numerical vector may be used itself as an input vector or may be concatenated with additional structured data information to be used as an input vector in the step of processing the downstream task.

In an exemplary implementation of the method, the number of encoders may be two or more, and a plurality of first numerical vectors output from each encoder may be concatenated to create one input numerical vector.

In an exemplary implementation of the method, N sequential chest radiology data may be passed through one encoder to obtain N sequential first numerical vectors.

In an exemplary implementation of the method, the method may provide analysis, diagnosis, or prediction of a specific disease based on the time-based result values obtained by processing the encoder or processing the encoder and the downstream task, or the time-based weighted average of the time-based result values after the chest radiology data is divided into certain time intervals.

In an exemplary implementation of the method, the method may include fixing weights of the network of the encoder when training the network of the downstream task, and then modifying (updating) the weights of the network of the downstream task through training, and then modifying (updating), through additional training, the entire weights of the networks of the encoder and the downstream task.

In an exemplary implementation of the method, the MLP having two or more fully connected layers may process the plurality of downstream tasks.

In an exemplary implementation of the method, the MLP may be trained through multi-task learning jointly with encoding network training of the encoder or may be separately trained after the encoder first completes the training.

In an exemplary implementation of the method, the MLP may receive additional structured data input information which is different from the first numerical vector, and the additional structured data input information corresponds to at least one or more of an age, a gender, vital signs (blood pressure, pulse, respiratory rate, body temperature, SpO2, and blood sugar), biosignals (e.g., ECG, PPG, EEG, and invasive pressure measurements of arteries and central veins), specimen test results (various blood tests, and biopsy), natural language information, and numerical or categorical data extracted from image data other than chest radiology data. The additional structured data input information may be concatenated with the first numerical vector or input separately from the first numerical vector.

In an exemplary implementation of the method, when the MLP predicts the occurrence of a specific disease, a marginal probability of a specific disease considering chest radiology data obtained while outputting the MLP and a marginal probability of a specific disease without considering the obtained chest radiology data may be presented together as a baseline risk probability, and how many times the marginal probability of a specific disease considering the obtained chest radiology data has increased in proportion to the marginal probability of a specific disease without considering the obtained chest radiology data may be displayed.

In an exemplary implementation of the method, the deep learning algorithm of the encoder corresponds to a vision network based on the structures of the CNN or the ViT. The structures of the CNN and the ViT may correspond to network structures commonly used for image data classification, and the classification performance and the efficiency thereof may be expanded through various modifications and extensions. In implementing the present application, selecting the CNN or the ViT of a specific structure belongs to a process optimized by the type and the amount of training data and the task being processed, and the encoder is not limited to the specific structure of the vision network, such as the CNN or the ViT.

In an exemplary implementation of the method, the deep learning algorithm of the encoder may be based on the CNN and may include an encoder subunit.

In an exemplary implementation of the method, the encoder subunit includes: one or more convolution layers; one or more fully connected layers, wherein the fully connected layers include a non-linear activation function; and an attention layer that summarizes the feature set extracted from the channel-specific chest radiology data to extract a representative value and recalibrates the channel-specific feature set to reflect a contribution of the channel-specific feature set based on the representative value, wherein the feature set includes a morphological feature for each channel, and the recalibrated channel-specific feature set may more focus on the morphological feature for each channel than the feature set.

In an exemplary implementation of the method, the one or more convolution layers may include a depthwise-separable convolution layer that individually convolutes the chest radiology data for each of the one or more channels.

In an exemplary implementation of the method, the attention layer may pool the feature set to summarize the feature set.

In an exemplary implementation of the method, the attention layer may be configured to calculate the channel-specific contribution by passing a channel-specific representative value through the fully connected layer, and recalibrate the channel-specific feature set by multiplying the channel-specific contribution by the feature set.

In an exemplary implementation of the method, the attention layer may be configured to calculate the channel-specific contribution by scaling a result of passing the channel-specific representative value through the fully connected layer into the numerical value within a specific range.

In an exemplary implementation of the method, the encoder subunit includes a squeeze-excitation layer that extracts an average for each channel to calculate a scalar value, wherein the scalar value for each channel is between 0 and 1. The encoder subunit is scaled according to the importance of the channel, passes a vector in which the scalar value for each channel are gathered through the fully connected layer, and then applies a sigmoid/RELU function to reduce the dimension.

In an exemplary implementation of the method, the encoder may include a plurality of convolution blocks, and the subunit may be included in a convolution block other than the first convolution layer.

In an exemplary implementation of the method, the convolution block includes a first encoder subunit and a second encoder subunit, wherein the first encoder subunit is applied closer to an input end than an output end of the convolution block, compared to the second encoder subunit. The attention layer may be configured to focus more on an operation of extracting a representative value by summarizing the feature set than the second encoder subunit, among the operation of extracting a representative value by summarizing the feature set and the operation of recalibrating the feature set according to the channel-specific contribution, wherein the representative value of the first encoder subunit reflects the morphological feature more than the representative value of the second encoder subunit. The second encoder subunit is applied closer to the output end than the input end of the convolution block, compared to the first encoder subunit. The attention layer may be configured to focus more on the operation of recalibrating the feature set according to the channel-specific contribution than the first encoder subunit, among the operation of extracting the representative value by summarizing the feature set and the operation of recalibrating the feature set according to the channel-specific contribution.

In an exemplary implementation of the method, the last convolution block of the encoder may further include a non-local network, wherein the non-local network may be configured to compare similarity between spatial points of the chest radiology data to implement spatial attention.

In an exemplary implementation, the analysis result includes disease prediction and diagnosis. When the analysis unit predicts or diagnoses a disease, the disease may include acute respiratory distress syndrome (ARDS), pneumonia, abscess, aspiration pneumonia, atypical pneumonia, active tuberculosis, non-tuberculous mycobacteria, chronic obstructive pulmonary disease (COPD), interstitial lung disease, bronchiectasis, sarcoidosis, lung nodule, lung mass, lung cancer, lung metastasis, aortic dissection, aortic aneurysm, pleural effusion, empyema, pneumothorax, pneumoperitoneum, pneumopericardium, pneumomediastinum, subcutaneous emphysema, coronary artery calcification, cardiomegaly, pulmonary edema, pericardial effusion, pulmonary embolism, chamber (LA, LV, RA, RV) enlargements, valvular (aortic, mitral, tricuspid, pulmonic) calcification/stenosis/regurgitation, hypertrophic cardiomyopathy, and various fractures, tumors, and metastasis of ribs, sternum, and spine.

In an exemplary implementation of the method, the analysis result may include disease diagnosis assistance information for determining whether the disease improved or worsened, using the first numerical vector. When the disease diagnosis assistance information is provided, the chest radiology data may be a plurality of pieces of chest radiology data measured at regular intervals. Each of the plurality of pieces of chest radiology data may pass through the pooling layer of the encoder to provide diagnosis assistance information on whether a disease improved or worsened from the obtained first numerical vector.

In an exemplary implementation of the method, the analysis result may include providing disease diagnosis assistance information. The chest radiology data is a plurality of pieces of chest radiology data measured at regular or irregular time intervals. The analysis unit may arrange each of the first numerical vectors of the plurality of pieces of chest radiology data as a sequential vector, may concatenate the sequential vectors in a vector length direction to pass through the MLP network, may concatenate the sequential vectors in a vertical direction to the vector length direction to pass through the transformer network, or may not concatenate the sequential vectors to sequentially pass through the RNN, and extract the second numerical vector by encoding information about time using a function to diagnose whether the patient's condition related to a specific disease improved or worsened.

In an exemplary implementation of the method, the encoder may perform training through supervised learning based on clinically defined morphological features of chest radiology data.

In an exemplary implementation of the method, the encoder may perform training through self-supervised learning using data obtained by modifying chest radiology data in a certain manner as training data.

In an exemplary implementation of the device, when the original chest radiology data and the augmented data obtained by subjecting the original chest radiology data to data augmentation are input to the encoder, the encoder may include a process of training the encoder so that each of the calculated first numerical vectors is the same or highly similar.

In an exemplary implementation of the method, the process of calibrating each of the calculated first numerical vectors to be the same or highly similar may minimize the distance of each of the calculated second numerical vectors.

On the other hand, in another aspect, an exemplary implementation provides a computer-readable recording medium for storing program instructions readable by a computer and operable by the computer, the program instructions, when executed by a processor of the computer, causing the processor to perform the foregoing method.

### Advantageous Effects

According to exemplary implementations of the present application, it is possible to extract numerical vectors from atypical chest radiology, in particular chest radiology data, and to utilize the same in various clinical situations.

In particular, it is possible to extract general numerical information that maximizes the utilization range of the chest radiology information while utilizing the existing clinical framework as it is. This general numerical information (embedding vector) is not only used as it is, but can also be fused and utilized with other information of the patient. In addition, the change in the patient's condition can be easily quantified by quantifying the chest radiology data. Accordingly, the general numerical information can be usefully used for initial evaluation and treatment response evaluation in a hospital room, an intensive care unit, and an emergency room. In addition, some of the structured numerical vectors are utilized as inputs to other artificial intelligence algorithms or medical protocols for various diagnoses that can be associated with the chest radiology data.

The effects of the present application are not limited to the effects mentioned above, and other effects not mentioned may be clearly understood by a person skilled in the art from the description of the claims.

### BRIEF DESCRIPTION OF THE DRAWINGS

In order to describe the exemplary embodiments of the present application more clearly, the drawings required for the description of the embodiments are briefly introduced below. It should be understood that the following drawings are for the purpose of describing embodiments of this specification only and are not intended to be limiting. In addition, some components may be shown with various modifications, such as exaggerations and omissions, in the following drawings for clarity of description.

FIG. 1 is a schematic diagram of a device for analyzing a disease by converting chest radiology data into numerical vectors, according to an embodiment of the present application.

FIG. 2 is a flowchart of a method of analyzing a disease by converting chest radiology data into numerical vectors, according to an embodiment of the present application.

FIG. 3 is a diagram illustrating a chest radiology encoder subunit, according to an embodiment of the present application.

FIG. 4 is a diagram illustrating a chest radiology encoder, according to an embodiment of the present application.

FIG. 5 is a diagram illustrating utilization of numerical vectors obtained from a plurality of pieces of chest radiology data obtained through repetitive measurement, according to another embodiment of the present application.

FIG. 6 is a diagram illustrating utilization of N sequentially obtained numerical vectors, according to another embodiment of the present application.

### DETAILED DESCRIPTION OF THE EMBODIMENTS

Hereinafter, some embodiments of the present application will be described in detail with reference to exemplary drawings. In adding reference numerals to components in each drawing, identical components may have the same numerals as much as possible even if they are shown in other drawings. In addition, when describing the present embodiments, if it is determined that a detailed description of a related known configuration or function may obscure the gist of the present technical idea, the detailed description thereof may be omitted.

### Definition of Terms

When “includes,” “has,” “consists of,” etc. mentioned in this specification are used, other parts may be added unless “only” is used. When a component is expressed in the singular, it can also include the plural, unless specifically stated otherwise.

Additionally, in describing the components of this application, terms such as first, second, A, B, (a), (b), etc. may be used. Unless otherwise specified, these terms are only used to distinguish the component from other components, and the nature, sequence, order, or number of the components are not limited by the term.

In this specification, “training” or “learning” is a term referring to performing machine learning through procedural computing.

In this specification, network refers to a neural network of a machine learning algorithm or model.

In this specification, the terms “unit,” “module,” “device,” or “system” are intended to refer to a combination of hardware as well as software driven by the hardware. For example, the hardware may be a data processing device including a central processing unit (CPU), a graphics processing unit (GPU), or other processor. In addition, software may refer to a running process, an object, an executable, a thread of execution, and a program.

In this specification, a numerical vector or numerical vector information herein refers to structured coordinate-based numerical data in a consistent structural and/or semantic form created through a deep learning algorithm for application to one or more machine learning tasks or operations, associated with (reflecting) features extracted from chest radiology data.

Converting specific data into numerical vectors refers to converting unstructured data having various formats and sizes, such as chest radiology images, into numerical vectors (or array) that is shorter (smaller) than the original and has a constant length (constant dimension and size in the case of format, array), and each of elements thereof has a consistent meaning for each position. This consistently represents where specific chest radiology data is located in the vector space defined by each element, and this abstract coordinate information can be utilized in various ways (algorithms) in various downstream tasks.

The chest radiology image, which is input data, may be input by resizing and cropping the same to a specific size or normalizing the same. The chest radiology image is a monochrome image, and the number of channels at the time of input is generally one, but a three-channel (or four-channel including an alpha channel) color image may also be input as multi-channel two-dimensional image data or converted into a monochrome image for input processing.

In the process of generating such a numerical vector, the features of a network structure (especially a network structure including a squeeze excitation, and a non-local network) to be presented herein may allow the encoder to apply an attention mechanism between various feature maps extracted from the chest radiology data and between different anatomical locations on a two-dimensional plane, thereby helping the generated numerical vector to encode a combination of various features of chest radiology.

The anatomical, physiological, and pathological features of chest radiology data are widely and efficiently reflected, and the features of the training method (assistant learning based on multitask learning) help to efficiently extract highly versatile features from various tasks in such a wide range of feature extraction processes.

This makes it possible to extract high-quality numerical vectors that make new downstream task training very easy, facilitating few-shot or one-shot learning.

In this specification, the numerical vectors may be separately represented as a first numerical vector, a second numerical vector, and the like. For example, the first numerical vector may refer to an output from the encoder using the deep learning algorithm, and the second numerical vector may refer to an output of the additional machine learning algorithm, such as a downstream task, using the first numerical vector. In some drawings, for example, sequential vectors included in the first numerical vector may be represented as vector 1, vector 2, vector 3, and the like.

Embedding herein may refer to the task or output (numerical vector itself) of converting chest radiology and unstructured data into the above-mentioned numerical vector.

In the present specification, the numerical vector having versatility means that the numerical vector can be used for machine learning for purposes other than a specific purpose, preferably for multiple machine learning purposes. That is, the numerical vector encompasses, preferably inclusively and/or efficiently encompasses, the morphological features of the particular chest radiology image, such that the numerical vector can be effectively utilized in unknown downstream tasks to be applied in the future or already being applied, preferably in two or more downstream tasks, and more preferably in most downstream tasks.

For example, in order to facilitate understanding, the numerical vector that is not versatile is taken as an example. If a numerical vector composed of 100 elements has 3 elements that are effective in diagnosing a specific disease, such as myocardial infarction, and the remaining 97 elements that have redundant or noisy information, then the numerical vector may not be utilized in the downstream task other than diagnosing myocardial infarction and may not have versatility. Several clinical diagnostic tasks may be performed simultaneously, rather than one diagnostic task, to fill the elements of the vector with meaningful information. However, this alone causes the numerical vector to encode only the features associated with the already trained diagnoses, making it difficult to apply to the unknown downstream task.

On the other hand, in the exemplary implementations of the present application, the squeeze excitation and non-local networks can improve the range and quality of the feature information to be included in the numerical vector as described above, thereby improving the versatility. Furthermore, in the exemplary implementations of the present application, it is possible to further increase the versatility of the numerical vector by additionally applying 1) supervised learning based on a previously clinically defined morphological feature, and 2) self-supervised learning for learning a morphological feature of chest radiology that is independent of clinical information. In addition, for efficient placement of information in the vector space defined by the numerical vector, 3) unsupervised learning, which will be described below, can be further performed to further increase the versatility of the numerical vector.

In this specification, unstructured data, which is a set of measured numerical data, may refer to data that 1) is not consistent in the size and/or number of dimensions, 2) is not consistent in the analysis of numerical values according to positions, or 3) has a large size or complexity and needs to be simply modified.

The structured data in this specification means that the number of dimensions and the size are constant. The structured data is consistent in the analysis of each numerical value according to positions and is not large in size (not excessively large in the number of elements) and is simpler than the structured data, making it possible to train the machine learning algorithm for the downstream task with only a small number of data, compared to the structured data. This may include, for example, chest radiology that has been embedded and converted into numerical vectors, and tabular data, such as the patient's age, gender, blood pressure, pulse rate, respiratory rate, and body temperature.

The downstream task herein may refer to one or more, in particular multiple, machine learning tasks using numerical vectors obtained through embedding. As described below, this may include 1) supervised learning, 2) unsupervised learning, 3) self-supervised learning, 4) clustering, and 5) anomaly detection.

As used herein, a method or a device for analyzing a disease may include analyzing a disease or health, predicting the disease or health, and providing diagnostic information about the disease.

### Description of Exemplary Implementations

In the exemplary implementations of the present application, the deep learning-based artificial intelligence algorithm is used for chest radiology image data to extract numerical vector information that can be variously used in various clinical situations, particularly versatile numerical vector information. The obtained numerical vectors can be used to estimate 1) lung abnormalities, 2) cardiac, macrovascular, mediastinal abnormalities, 3) musculoskeletal abnormalities, 4) major clinical diagnoses, 5) presence or absence of major devices, 6) major clinical events, and 7) the need for major clinical treatments individually or collectively. Each of the specific examples is as follows, and each classification is not mutually exclusive:
1) Lung abnormalities: consolidation, infiltration, cavitation, atelectasis, pneumonectomy, lobectomy, segmentectomy;
2) Cardiac, macrovascular, mediastinal abnormalities: cardiomegaly, mediastinal enlargement, aortic calcification, coronary calcification;
3) Musculoskeletal abnormalities: fracture, lytic (solubility), sclerotic (curability);
4) Major clinical diagnosis: acute respiratory distress syndrome (ARDS), pneumonia, abscess, aspiration pneumonia, atypical pneumonia, active tuberculosis, non-tuberculous mycobacteria, chronic obstructive pulmonary disease (COPD), interstitial lung disease, bronchiectasis, sarcoidosis, lung nodule, lung mass, lung cancer, lung metastasis, aortic dissection, aortic aneurysm, pleural effusion, empyema, pneumothorax, pneumoperitoneum, pneumopericardium, pneumomediastinum, subcutaneous emphysema, coronary artery calcification, cardiomegaly, pulmonary edema, pericardial effusion, pulmonary embolism, chamber (LA, LV, RA, RV) enlargements, valvular (aortic, mitral, tricuspid, pulmonic) calcification/stenosis/regurgitation, hypertrophic cardiomyopathy, and various fractures, tumors, and metastasis of ribs, sternum, and spine;
5) Presence or absence of major devices: central vein catheter, peripheral inserted central catheter (PICC), pacemaker, implantable cardioverter defibrillator (ICD), chest tube, percutaneous drainage, nasogastric tube; and
6) Major clinical events: shock, respiratory failure, cardiac arrest, endotracheal intubation, mechanical ventilation.

In addition to chest radiology data, other structured information (age, gender, blood pressure, pulse rate, respiratory rate, body temperature, laboratory test results) and unstructured information (chief complaint, underlying disease, text, various kinds of radiation and ultrasound image information, acoustic information such as auscultation sound, and various kinds of biosignals) that have been appropriately modified can also be further concatenated into the corresponding numerical vectors to increase the accuracy of diagnosis.

The algorithm of the exemplary implementations of the present application may include a deep learning algorithm part, such as a modified convolutional neural network (CNN) and a visual transformer (ViT), and/or an algorithm part that processes additional information other than chest radiology data.

In addition, in the exemplary implementations of the present application, the chest radiology data may be obtained to provide the assistance information regarding analysis, prediction, and diagnosis of a disease.

In exemplary implementations, a device for converting chest radiology data into numerical vectors may include: an acquisition unit for acquiring chest radiology data; and an encoder that receives the chest radiology data and uses a deep learning algorithm so as to calculate a numerical vector (which may be referred to as a first numerical vector).

In exemplary implementations, a device for analyzing a disease by converting chest radiology data into numerical vectors includes: an acquisition unit for acquiring chest radiology data from a chest radiology measurement device; an encoder that receives the chest radiology data and uses a deep learning algorithm so as to calculate a numerical vector (which may be referred to as a first numerical vector); and an analysis unit that uses the numerical vector so as to provide disease-related analysis, prediction, or diagnosis.

In exemplary implementations, a method of converting chest radiology data into numerical vectors, performed by a processor, includes: obtaining chest radiology data from a chest radiology measurement device; inputting the chest radiology data to an encoder; and calculating a numerical vector (which may be referred to as a first numerical vector) using a deep learning algorithm through the encoder.

In exemplary implementations, a method of analyzing a disease from chest radiology data using deep learning, performed by a processor, includes: obtaining chest radiology data from a chest radiology measurement device; inputting the chest radiology data into an encoder; calculating a numerical vector (which may be referred to as a first numerical vector) using a deep learning algorithm through the encoder; and providing disease-related analysis, prediction, or diagnosis information using the numerical vector.

In exemplary implementations of chest radiology analysis, the numerical vector may be used simultaneously in a downstream task.

As such, since the numerical vector is configured to perform multiple tasks simultaneously, error signals from each output end of the network of the downstream task are gathered at one encoder end to train one encoder when backpropagated. Accordingly, the numerical vector can be a numerical vector with improved versatility.

In an exemplary implementation, the first numerical vector may be used as an input vector of the network of the downstream task by itself or concatenated with additional structured data information.

The additional structured data information may include at least one of: existing structured data information, such as age, gender, vital signs (blood pressure, pulse rate, body temperature, respiratory rate, and oxygen saturation), and various laboratory test results; unstructured data information converted into structured data information through a machine learning method (e.g., images, sounds, and biosignals (which are different from chest radiology input to the encoder to obtain the first numerical vector); and natural language information, such as symptoms, diagnosis names, medical records, and the like transformed into structured data information through natural language processing.

In an exemplary implementation, the number of encoders may be two or more, and a plurality of first numerical vectors output from each encoder may be concatenated to create one input numerical vector. The input numerical vector may be set as an input value of the network of the downstream task, and a diagnosis to be predicted may be set as the output value of the network of the downstream task to train the network.

In an exemplary implementation, N sequential chest radiology data may be passed through one encoder to obtain N sequential first numerical vectors. These N sequential first numerical vectors may be used as input values for learning of the network of the downstream task that predicts whether a particular disease improves or worsens over time, predicts a risk of a particular disease, or predicts occurrence of a clinical event.

In an exemplary implementation, the device may may provide analysis, diagnosis, or prediction of a specific disease based on the time-based result values obtained by processing the encoder or processing the encoder and the downstream task, or the time-based weighted average of the time-based result values after the chest radiology data is divided into certain time intervals.

In an exemplary implementation, the device for converting chest radiology data into numerical vectors or the device for analyzing a disease includes a downstream task processing unit or a processing step of processing a downstream task by utilizing numerical vectors, wherein the downstream task may process multiple tasks, and each of the tasks may be performed by a multi-layer perceptron (MLP) having two or more fully connected layers.

In an exemplary implementation, when the MLP predicts the occurrence or non-occurrence of a specific disease, a marginal probability of the disease considering chest radiology data and a marginal probability of the disease without considering chest radiology data may be presented together as a baseline risk probability while outputting the MLP, and how many times the probability of the disease occurrence considering the chest radiology data is increased in proportion to the probability without considering the chest radiology data may be displayed.

In an exemplary implementation, the MLP for each task may be trained jointly with encoding network training of the encoder, or separately after the encoder first completes the training.

The deep learning algorithm of the encoder corresponds to a convolution neural network (CNN) or a vision network based on a transformer (visual transformer: ViT) structure. The structures of CNN and ViT may correspond to network structures commonly used for image data classification, and the classification performance and the efficiency thereof may be expanded through various modifications and extensions. In implementing the present application, selecting the CNN or the ViT of a specific structure belongs to a process optimized by the type and the amount of training data and the task being processed. The encoder is not limited to the specific structure of the vision network, such as the CNN or the ViT.

In an exemplary implementation, the encoder is based on the CNN and includes an encoder subunit. The encoder subunit may include a depthwise-separable convolution layer that independently convolutes the chest radiology data on a channel-by-channel basis.

In an exemplary implementation, the encoder subunit applies a squeeze-excitation mechanism to extract one numerical value (an average or a highest value) for each channel. The resulting numerical vector is passed through a network consisting of two or more fully connected layers containing a non-linear activation function, such as RELU, and then a sigmoid function is applied to obtain numerical values between 0 and 1 for each channel. The numerical values are each multiplied by the corresponding channel to recalibrate the features for each channel.

In an exemplary implementation, the encoder may include a first convolution layer and a plurality of convolution blocks each including a plurality of encoder subunits.

In an exemplary implementation, the last convolution block of the encoder may further include a non-local network. The non-local network (or non-local neural network) uses features of all positions of the input data when encoding information of a specific position (a spatial point on the chest radiology feature map). In this process, each position has a different degree of contribution, and the degree of contribution is determined through the attention mechanism.

In an exemplary implementation, the MLP for each task may receive additional structured data input information different from the numerical vectors output by the encoder. The additional input information may include at least one of an age, a gender, vital signs (blood pressure, pulse rate, body temperature, respiratory rate, accompanying symptoms, and oxygen saturation), various laboratory test results, and unstructured data (image, sound, biosignals) converted into structured numerical information.

In exemplary implementations, the device described above may be chest radiology measurement equipment, storage equipment, or interpretation equipment. Examples thereof include, but are not limited to, various chest radiology equipment (including both fixed and mobile), medical image storage servers and viewers (e.g., PACS), electronic health records, an application programming interface (API) service for medical information, software that can accept and analyze chest radiology data through a camera or scan equipment (smartphones, desktops, augmented reality glasses), and the like.

In addition, in exemplary implementations, there is provided a computer-readable recording medium that is readable by a computer and that stores program instructions operable by the computer, the program instructions, when executed by a processor of the computer, causing the processor to perform the method of converting chest radiology data from the chest radiology data into numerical vectors.

### DESCRIPTION OF PREFERRED EMBODIMENTS

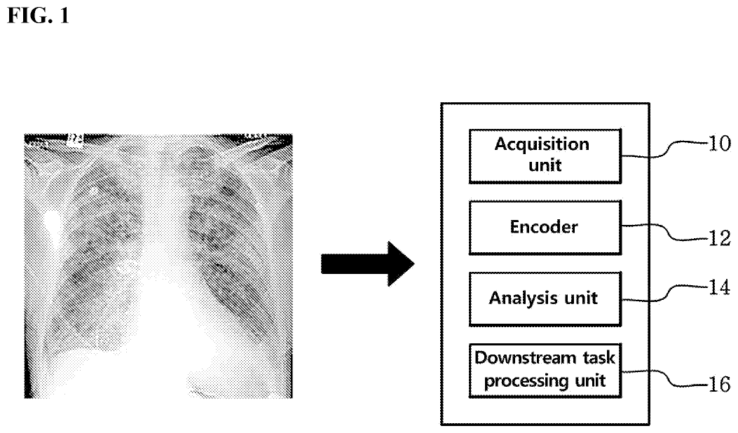

FIG. 1 is a schematic diagram of a device for analyzing a disease 1 (Hereinafter, referred to as “device 1”) by converting chest radiology data into numerical vectors according to an embodiment of the present application.

Referring to FIG. 1, the device 1 according to an embodiment of the present application includes: an acquisition unit 10 for acquiring chest radiology data from a chest radiology measurement device; an encoder 12 that receives the chest radiology data and uses deep learning to calculate a numerical vector; an analysis unit 14 that uses the numerical vector calculated by the encoder to provide an analysis result that is information regarding disease-related analysis, prediction, or diagnosis; and one or more downstream processing units 16 that utilize the numerical vector to process a downstream task. Although FIG. 1 shows the downstream processing unit 16 separately from the analysis unit 14, the downstream processing unit 16 may be included as part of the analysis unit 14 or may replace the analysis unit 14.

The acquisition unit 10 may acquire a chest radiology image from a chest radiology measurement device that is attached to a body part of a subject and measures the chest radiology image of the subject (user). The encoder 12, which is a computing device including a processor, may receive chest radiology data as an input from the acquisition unit 10, analyze the chest radiology data, and apply an attention mechanism between various feature maps and anatomical positions to generate various feature maps and calculate a numerical vector by pooling the same. The numerical vector may then be utilized to provide analysis, prediction, and diagnosis information for various diseases through the analysis unit 14 or the downstream processing unit 16.

In an embodiment, the encoder 12 may include various computing devices including, for example, a computer such as a personal computer (PC) or a notebook, a smartphone, a server, and the like.

In an embodiment, the encoder 12 may be implemented as a server, and the chest radiology data input to the encoder may be performed through a device (e.g., a user terminal or signal input equipment) connected to the server.

In this case, the server, including a plurality of computer systems or computer software implemented as a network server, may provide various pieces of information by configuring a website. The network server refers to a computer system and computer software (network server program) connected to a subordinate device capable of communicating with another network server through a computer network, such as a private intranet or the Internet, to receive a request to perform a task and then perform the task, thereby providing a performance result. However, in addition to such a network server program, it should be understood that the network server includes a series of applications operating on the network server and, in some cases, various databases built therein. For example, when various databases are included, the encoder 12 is configured to use external database information, such as a cloud. In this case, the encoder 12 may connect to an external database server (e.g., a cloud server) to perform data communication according to an operation.

In an embodiment, the encoder 12 for calculating a numerical vector may include a deep learning model. The deep learning model automatically trains features of respective chest radiology data by learning a large amount of unstructured chest radiology data in a deep neural network composed of a multi-layer network, and training a network for calculating a numerical vector by minimizing an error in an objective function, that is, prediction accuracy.

In an embodiment, the deep learning algorithm of the encoder corresponds to a convolutional neural network (CNN) or a vision network based on a transformer (visual transformer: ViT) structure. The structures of CNN and ViT correspond to network structures commonly used for image data classification, and the classification performance and the efficiency thereof may be expanded through various modifications and extensions. In implementing the present application, selecting the CNN or the ViT of a specific structure belongs to a process optimized by the type and the amount of training data and the task being processed, and the encoder is not limited to the specific structure of the vision network, such as the CNN or the ViT.

In an embodiment, the modified CNN structure applied to the encoder 12 in the present application is particularly more suitable for chest radiology analysis for the following reasons:
1) Use of squeeze excitation network: The squeeze excitation network effectively reflects the morphological information of each channel in the numeric vector extracted through the encoder, thereby improving the quality of the encoder and the numeric vector obtained by the encoder. Since morphological patterns (e.g., texture of lung lesions) are very important in the analysis of chest radiology, in extracting a large number of clinically meaningful numerical features from chest radiology, each numerical value should be non-linearly aggregated by selecting appropriate information for a specific morphological pattern that the numerical value needs to reflect. The squeeze excitation makes this possible by optimizing the contribution of each channel through the recalibration process described above when creating a representation provided in the next layer. Specifically, when applied to the side closer to the input end, the feature extraction focusing on a specific channel is performed according to the morphological pattern of the tissue, for example, classification of pneumonia and pulmonary edema according to the morphological texture of the lung parenchyma, and when applied to the end closer to the output end, nonlinearly aggregated abstract clinical information is selected.
2) Use of non-local network (or non-local neural network): The non-local network allows the numerical vectors extracted through the encoder to effectively reflect the interaction between the chest radiology features that are separated from each other in time, thereby improving the quality of the encoder and the numerical vectors obtained by the encoder. When interpreting the clinical meaning of chest radiology input at a specific time point, chest radiology data before and after the specific time point should also be considered. In addition, in order to apply such a process of referencing and integrating information to remote data, a network capable of learning how appropriate it is to integrate remote information (features) into current location information to be analyzed is required, wherein the non-local network described above performs this role.
3) Use of skip connection: The encoder 12 of the embodiment of the present application utilizes a deep learning structure that undergoes multiple layers of nonlinear transformation of input data. In this case, gradient vanishing may occur in which the loss signal of the output terminal is not sufficiently transmitted to the input terminal. The skip connection effectively reduces this problem. In addition, the skip connection allows the information of the input end to be reflected in the output end with minimal modification, so that the extracted numerical vector broadly reflects various features in the transformation process of the encoder, thereby improving the quality of the numerical vector.
4) Multi-task learning: The multi-task learning is one way to train an encoder network having the network features mentioned above, which helps to make one numerical vector obtained through the encoder versatile by allowing the numerical vector to be commonly used in several downstream tasks during the training process. As described above, the encoder 12 of the present application outputs the reduced numerical vector of a fixed size and format through an embedding process, which is utilized to perform various downstream tasks. The numerical vector used herein is used as input information of various machine learning algorithms for various purposes, and thus it is necessary to extract the comprehensive clinical state of the patient as efficiently as possible. Since the output vector of the encoder 12 is configured to perform multiple tasks to be described below simultaneously in the embodiment of the present application, when error signals from each output end of the network of the downstream task are backpropagated, the error signals are gathered at one encoder end to train one encoder, so that the encoder trained in this way can generate a versatile embedding vector that achieves the above-mentioned goal.

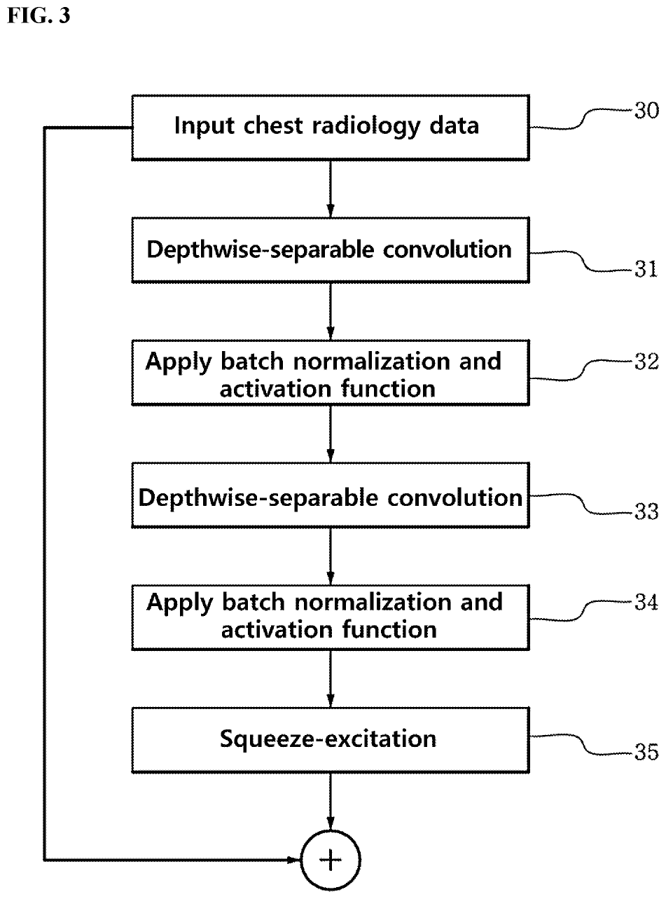

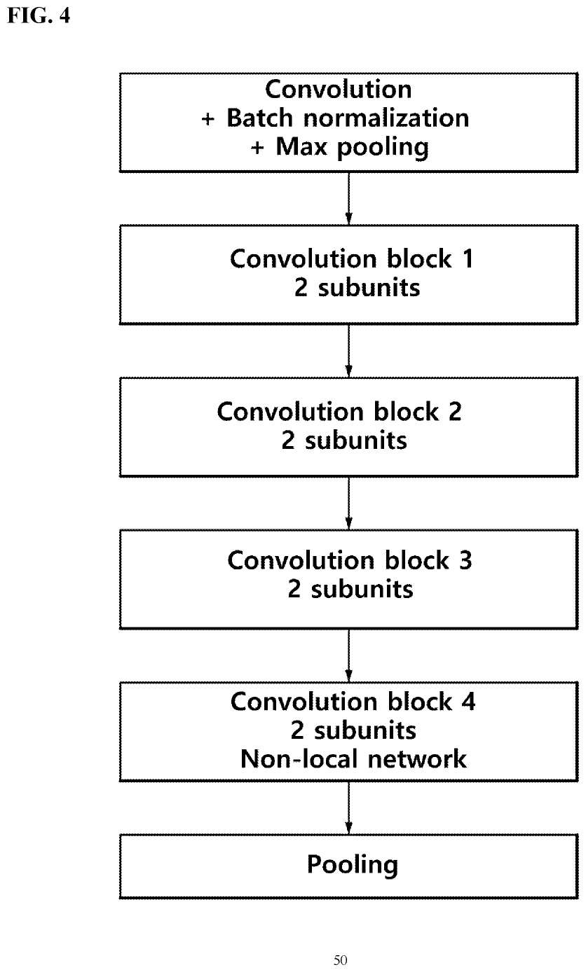

In one embodiment, the encoder 12 includes one convolution layer and a plurality of convolution blocks contiguous thereto, wherein each of the convolution blocks may include a plurality of contiguous chest radiology subunits. The encoder 12 may convert the chest radiology data into numerical vectors through the first convolution layer and the plurality of convolution blocks. A process of converting chest radiology data into numerical vectors by the encoder will be described in more detail with reference to FIGS. 3 and 4 below.

In an embodiment, the analysis unit 14 uses the numerical vector calculated by the encoder 12 to provide an analysis result, which is information about disease-related analysis, prediction, or diagnosis.

The analysis result of the analysis unit 14 includes disease prediction and diagnosis, and when the analysis unit predicts or diagnoses a disease, the disease may include acute respiratory distress syndrome (ARDS), pneumonia, abscess, aspiration pneumonia, atypical pneumonia, active tuberculosis, non-tuberculous mycobacteria, chronic obstructive pulmonary disease (COPD), interstitial lung disease, bronchiectasis, sarcoidosis, lung nodule, lung mass, lung cancer, lung metastasis, aortic dissection, aortic aneurysm, pleural effusion, empyema, pneumothorax, pneumoperitoneum, pneumopericardium, pneumomediastinum, subcutaneous emphysema, coronary artery calcification, cardiomegaly, pulmonary edema, pericardial effusion, pulmonary embolism, chamber (LA, LV, RA, RV) enlargements, valvular (aortic, mitral, tricuspid, pulmonic) calcification/stenosis/regurgitation, hypertrophic cardiomyopathy, various fractures, tumors, and metastasis of ribs, sternum, and spine. The analysis result of the analysis unit 14 includes a disease diagnosis, and when a disease is diagnosed, the analysis result may include heart rhythm abnormalities (tachycardia, bradycardia, and various arrhythmias) and heart structure and function abnormalities (heart failure, pericardial tamponade, valvular stenosis/failure, pulmonary hypertension, pulmonary embolism, and cardiomyopathy).

In one embodiment, the chest radiology data may be a plurality of pieces of chest radiology data measured at regular or irregular time intervals. Each of the pieces of chest radiology data may pass through the encoder, and each numerical vector thereof may be obtained to obtain a diagnosis from the analysis unit 14, or a plurality of numerical vectors may be simultaneously input into one machine learning algorithm to diagnose the disease or diagnose whether the disease improved or worsened.

The analysis unit 14 may arrange each numerical vector obtained from the plurality of pieces of chest radiology data as a sequential vector. When processing multiple numerical vectors as inputs, the multiple numerical vectors may be concatenated in the vector length direction to be converted into one input and passed through one multilayer perceptron (MLP) network, may be concatenated in the vertical direction to the vector length direction and passed through one transformer network, or may be sequentially passed through one recurrent neural network (RNN) without being concatenated according to an inspection execution order. In this case, information about time may be encoded using a function and may be concatenated with each of the input numerical vectors to increase accuracy.

Meanwhile, in embodiments, the downstream task processing unit 16 processes the downstream task by utilizing the numerical vector calculated by the encoder. In one embodiment, each task of the downstream task may be performed by the MLP having two or more fully connected layers.

In an embodiment, each task-specific MLP network may be trained together with an encoder network or separately from the encoder network after the encoder 12 first completes the training. When there are a plurality of networks of the downstream task, the networks are trained simultaneously through multi-task learning. The networks of the downstream task may receive additional structured data input information other than the numerical vector output from the encoder 12 to improve prediction accuracy, and the additional structured data input information may be concatenated into the numerical vector or processed through another input network. The additional structured data input information corresponds to at least one or more pieces of numerical or categorical data extracted from an age, a gender, vital signs (blood pressure, pulse, respiratory rate, body temperature, SpO2, blood sugar, and the like), biosignals (e.g., electrocardiogram (ECG), photoplethysmography (PPG), electroencephalogram (EEG), invasive pressure measurement values of arteries and central veins), specimen test results (various blood tests, and biopsy), natural language information, image data other than chest radiology data, and the like.

In one embodiment, the device 1 may be combined with automated assessment equipment (e.g., chest radiology, storage, analysis equipment) that may directly analyze ECG images obtained retrospectively/prospectively from a patient, e.g., in real time, to provide clinical information.

As one non-limiting example, the device 1 may be, but is not limited to, fixed or mobile X-ray imaging equipment, picture archiving and communication system (PACS), electronic health record (EHR), medical artificial intelligence software embedded in a smart device based on camera input, and the like.

Furthermore, in an embodiment, the device 1 may be combined with a chest radiology analysis device that provides clinical information by directly analyzing an image of visualized chest radiology output on paper or images from the obtained chest radiology.

As one non-limiting example, the device 1 may be, but is not limited to, a device equipped with an app, an EHR system using a camera or scanning equipment and equipped with an analysis algorithm, and the like.

Meanwhile, a method of converting chest radiology data into numerical vectors (“numerical vector conversion method”) is performed by a computing device including a processor. The computing device including the processor may be performed by, for example, the device 1 or at least some components thereof (e.g., the acquisition unit 10, the encoder 12, the analysis unit 14, and/or the downstream task processing unit 16, wherein the downstream task processing unit 16 may be present separately or may be included in the analysis unit 14), or may be performed by another computing device. Hereinafter, for clarity of description, the present application will be described in more detail with embodiments in which the method of converting numerical vectors is performed by the device 1 for converting the chest radiology data into numerical vectors.

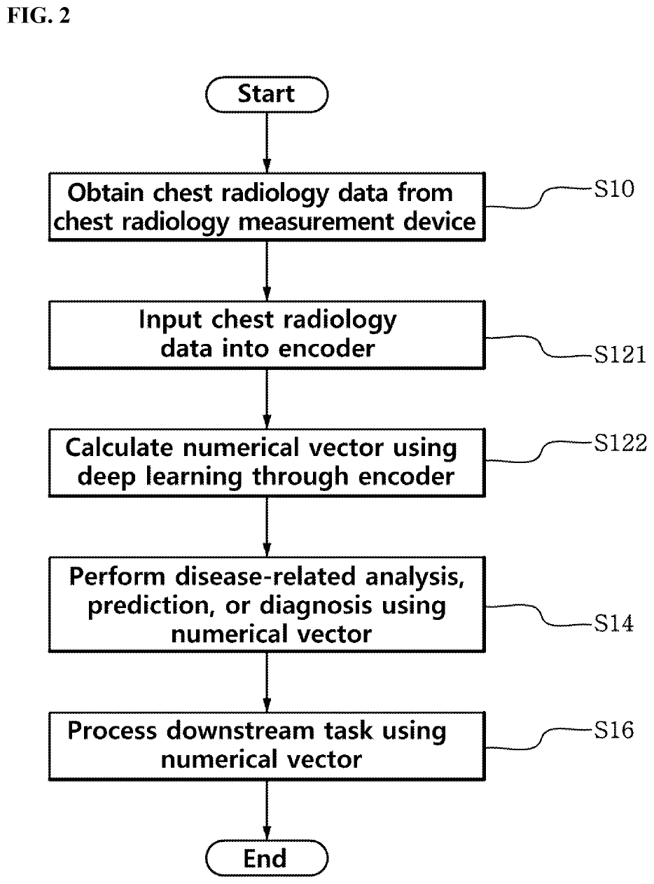

FIG. 2 is a flowchart of a method of analyzing a disease by converting chest radiology data into numerical vectors, according to an embodiment of the present application. Referring to FIG. 2, the method of analyzing a disease, performed by a processor, is a method of analyzing the disease from chest radiology data (CXR) using deep learning, the method including: obtaining (e.g., by the acquisition unit 10) chest radiology data from a chest radiology measurement device (S10); inputting (e.g., by the encoder 12) the chest radiology data into the encoder (S121); calculating a numerical vector using deep learning through the encoder (S122); and performing (e.g., by the analysis unit 14) disease-related analysis, prediction, or diagnosis using the numerical vector (S14). For example, the method further includes step S16 of processing a downstream task by using the numerical vector additionally by the downstream processing unit 16 or as part of the analysis step S14 by the analysis unit 14, and each task may be performed by the MLP having two or more fully connected layers.

FIG. 3 is a diagram illustrating an encoder subunit, according to an embodiment of the present application.

Referring to FIG. 3, in an embodiment, the encoder 12 is based on a CNN and includes a plurality of convolution blocks.

The encoder 12 includes the encoder subunit.

The ECG subunit is included in the remaining convolution blocks except for the first convolution layer.

The encoder subunit may include a depthwise-separable convolution layer that independently convolutes the channel-specific chest radiology data.

In the encoder subunit constituting the encoder 12, the chest radiology data (chest radiology image) passes through a depthwise-separable convolution layer twice and is input as input data of a next convolution layer through skip connection. The depthwise-separable convolution is a form in which depthwise convolution is followed by point-wise convolution.

FIG. 4 is a diagram illustrating an encoder, according to an embodiment of the present application. Referring to FIG. 4, in an embodiment, the encoder may include a convolution layer of an input end and four or more convolution blocks subsequent thereto. When the input data is a chest radiology image, the first convolution layer has 64 channel outputs. Then, after passing through a batch normalization layer and a max pooling layer, four convolution blocks are sequentially passed. Each convolution block includes two sequential encoder subunits, and the last block may include a non-local network. When all the blocks are passed, a global pooling process is finally performed. The kernel size, the stride size, the padding method and the number of output channels of all convolution and pooling layers, and the number of blocks, the number of subunits for each block, and the arrangement of the non-local network, which are objects of optimization, may be determined using various optimization methods (e.g., grid, random, Bayesian optimization method).

In an embodiment, each encoder subunit, having a structure having a series of processing processes, such as a depthwise-separable convolution layer (e.g., stride 2), a batch normalization layer, a depthwise-separable convolution layer (e.g., stride 1), a batch normalization layer, and a squeeze excitation layer, and may include one skip connection added to the result vector bypassing the series of processing processes, with reference to FIG. 3.

The squeeze-excitation is a methodology in which the compression of the feature map and the scale through recalibration are key, which focuses on channel relationships and explicitly models interdependencies between channels to adaptively recalibrate channel-specific characteristic responses.

In an embodiment, the last convolution block of the encoder may further include a non-local network. The non-local network adds an attention mechanism in a spatial manner. When an inner product value between a query vector of a specific spatial point of the feature map and a key vector of all spatial points is obtained and normalized through a softmax operation, a scalar value corresponding to a weight between 0 and 1 for each position of the feature map may be obtained. By multiplying each thereof by a value vector of the corresponding spatial point and adding the same all up, the value vectors of the specific spatial points may be converted into a weighted sum of the value vectors of all spatial points. The original feature map is added to the converted value through the skip connection to form an output value. The vectors corresponding to the above key, query, and value use those calculated using each independent parameter function from the input feature map. This process makes it possible to more efficiently determine the overall context of the chest radiology data by allowing signals from other distant points in time to be considered together when analyzing features at specific points in time of the chest radiology data (corresponding to specific positions of the one-dimensional input data). In contrast, a typical CNN has a limitation in that it only computes a local neighborhood. Even with Atrous convolution or a large kernel size, the area that the filter can see at once is limited. Operations that can only know local information along such a time axis or spatial axis usually perform iterative operations in order to obtain a global view. However, such iterative operations are inefficient and difficult to optimize, and multi-hop dependency occurs when modeling. The non-local network used in the present application overcomes these constraints by allowing reference between various feature combinations in the form of a weighted sum. In the embodiment of the present application, the non-local network is used by adding the same to the last convolution block of the encoder, but the arrangement thereof varies depending on the input data and the intended use.

In an additional embodiment, a plurality of different encoding networks trained through various settings may be gathered and used together, and the encoder described above may use various numbers and formats of depthwise-separable convolution layers in each convolution layer according to input signals, processing problems, and analysis equipment. In addition, a kernel size, a stride size, a padding method, an output size, and the like may be variously set for each convolution layer. In this case, multiple embedding vectors may be extracted from one piece of chest radiology data, and these results can be aggregated (e.g., concatenation, addition, attention mechanism) and used for prediction and diagnosis of diseases.

In one embodiment, the input data is input by resizing and cropping the chest radiology image to a particular size, and normalizing the same. Since the input data is monochrome, the number of channels at the time of input is generally one, but a three-channel (or four-channel including an alpha channel) color image may also be input as multi-channel two-dimensional image data or converted into a monochrome image for input processing. The kernels of all convolution layers and depthwise-separable convolution layers are two-dimensional. The kernels of all pooling layers (max pooling and global average pooling) are two-dimensional. The output after the last pooling (e.g. global average pooling) is an N×D or D dimensional vector. In one embodiment, a numerical vector value calculated by the encoder may be utilized for the downstream task. Each task is performed by the MLP having two or more fully connected layers. The MLP for each task may be trained 1) jointly with the encoder, or 2) independently by receiving an embedding vector output by the encoder 12 that has completed the training in advance as an input. If the training is performed through the latter method, only the downstream task MLP is trained while the weight values of the encoder are fixed, and after the training is completed, a fine tuning process may be added in which the weight fixing of the weight values of the encoder is released and the entire network is additionally trained through backpropagation.

In an embodiment, the MLP for each task may receive additional structured data input information which is different from the calculated numerical vector of the encoder 12 to increase prediction accuracy. The additional input information may be preprocessed, such as standardization, and then concatenated into the numerical vector or may be processed through another input network and then concatenated to be processed as an input.

When the output values of the MLP are used as they are in the case of a multivariate regression analysis problem (predicting various numerical values).

In the case of a classification problem (selecting one of several items), the probability of being included in each item is obtained by passing through a softmax function, and an item having the highest probability is selected.

In the case of a problem of predicting the occurrence or non-occurrence of certain events, each of the output values is passed through a sigmoid function, which is interpreted as a probability of the occurrence of the event. This probability can be regarded as a conditional probability obtained by analyzing the chest radiology data. When outputting the same, a marginal probability without considering the input chest radiology data is presented together as a baseline risk probability, and a diagram (e.g., a bar graph) that visually shows how many times this probability has increased in proportion (conditional probability/marginal probability) by the chest radiology data may be displayed.

In one embodiment, an exemplary downstream task included in the present application is a clinical diagnosis or prediction task of a disease, wherein the disease may include acute respiratory distress syndrome (ARDS), pneumonia, abscess, aspiration pneumonia, atypical pneumonia, active tuberculosis, non-tuberculous mycobacteria, chronic obstructive pulmonary disease (COPD), interstitial lung disease, bronchiectasis, sarcoidosis, lung nodule, lung mass, lung cancer, lung metastasis, aortic dissection, aortic aneurysm, pleural effusion, empyema, pneumothorax, pneumoperitoneum, pneumopericardium, pneumomediastinum, subcutaneous emphysema, coronary artery calcification, cardiomegaly, pulmonary edema, pericardial effusion, pulmonary embolism, chamber (LA, LV, RA, RV) enlargements, valvular (aortic, mitral, tricuspid, pulmonic) calcification/stenosis/regurgitation, hypertrophic cardiomyopathy, and various fractures, tumors, and metastasis of ribs, sternum, spine.

To this end, in addition to the chest radiology, additional structured data information may be received as an input, and the additional structured data input information corresponds to an age, a gender, structured biometric information (blood pressure, pulse rate, respiratory rate, body temperature, laboratory test results), and unstructured information (chief complaint, underlying disease, text, ultrasound image information, acoustic information such as auscultation sound, and various biosignals) structured through appropriate modification.

In the embodiments of the present application, in order to improve the numerical vector (embedding) quality of the encoder, three auxiliary learning tasks (supervised learning/self-supervised learning/unsupervised learning) may be applied in the process of training the encoder.

First, the supervised learning may be performed in parallel with the downstream task. This may include the technical characteristics of chest radiology image (imaging modality-PA, AP, lateral and imaging-related parameters-energy and duration of exposure), the characteristics of the imaging subject (age, sex, height, weight, underlying disease), the morphological features of radiology images that assist in diagnosing a disease, such as consolidation, infiltration, cavitation, atelectasis, airway deviation, air-fluid level, nodule, nodular pattern, reticular pattern, honeycombing, ground glass pattern, increased cardio-thoracic ratio, mediastinal enlargement, coronary calcification, presence or absence of A-line and B-line, increased interstitial marking, and the like. In addition, all parameters that quantify and show the quantified results (FVC, FEV, TV, MV, TLC, RV, FEF, PEFR) of pulmonary function tests performed together at close time points and the increase or decrease thereof, or the ECG test results (left ventricular function, right ventricular functions, pericardial effusion, left/right atrial size, left/right ventricular size, pulmonary hypertension) performed together at near time points, and the presence or absence of abnormalities thereof are included in the auxiliary learning tasks. This task based on supervised learning allows already clinically well-defined morphological or clinical features to be reflected in the numerical vector, thereby improving the quality of the numerical vector.

For reference, the learning contents are mainly defined by medical practitioners or clinicians by extracting a morphological pattern observed in chest radiology or correspond to clinical information provided through examinations performed together at a near point in time. These learning contents cannot be the final diagnosis by themselves. However, the numerical vector (embedding) quality of the encoder network is improved in the process of training to learn the above contents. In addition, since the auxiliary learning tasks by the supervised learning may also be usefully used in clinical practice, the trained network result may be output and utilized in clinical decision making.

Second, the self-supervised learning may be performed in parallel with the downstream task. This includes, after modifying the original chest radiology data in a specific way (image augmentation), 1) a method of inferring the type (and content) of the modification, and 2) a method of reconstructing the original using the modified input. The modifications used in the above method 1) may vary, including such as i) adding various noises of the original image, ii) randomly changing a set value (brightness, saturation, contrast) of the entire image, iii) cutting out and discarding a specific section(s) of the image, or selecting only a specific region and discarding the rest, and iv) cutting out and randomly reconstructing the image. These modifications may be applied one by one or in multiples. The main task is to estimate which modifications (or combinations) have been applied and may sometimes be trained to infer the specific content of the modifications. Similar image variations can be used in the above method 2). This self-supervised learning task allows the numerical vector to better reflect the morphological features of chest radiology, allowing extraction of high quality numerical vectors.

Third, the unsupervised learning may be performed in parallel with the downstream task. The unsupervised learning content applied in the present application is as follows. The network training procedure of the present application applies the data augmentation procedure as described above. In this process, N modified chest radiology input data are generated from one piece of chest radiology data. In this case, if the original chest radiology is M, M×N chest radiology input values are generated. When two chest radiology are extracted from these M×N chest radiology, if the two chest radiology have the same original, the numerical vectors generated therefrom should be the same or very similar. In order to satisfy this constraint, in the present unsupervised learning task, the following loss term that minimizes the distance on two augmented data points from the same original is added to the existing loss function.

β·I(VecA and VecB is originated from same CXR image)·∥VecA−VecB∥

β is an optionally adjustable hyper-parameter, I is an indicator function, and μVecA−VecB∥ refers to the distance of two vectors. As an example, the distance measurement method may use the Euclidean distance, but is not limited thereto. Th distance measurement method may be changed in the same manner as the β value according to each problem situation. The addition of such a loss term trains the encoder to place each numerical vector closer to the vector space obtained from the numerical vector as they have similar shapes and each numerical vector is efficiently placed in the vector space defined by the numerical vector, resulting in an improvement in the embedding quality of the numerical vector.

When the auxiliary learning tasks based on supervised/self-supervised/unsupervised learning are performed as described above, the network of the downstream task for learning is trained jointly with an encoder network, which may be independently performed prior to training of the downstream network for clinical diagnosis/prediction or may be performed simultaneously with training of the clinical diagnosis/predictions network. If the pretraining is done, the weight of the encoder is fixed after the pretraining, and only the clinical diagnosis/prediction network is trained. Then, if necessary, the weight of the encoder is unfixed, and a fine tuning process is applied to train both the encoder and the network of the downstream task for clinical diagnosis/predictions simultaneously. If the self-supervised learning network and the clinical diagnosis/prediction network are trained simultaneously, the weight update is made throughout the weights of all networks including the encoder.

The prior/parallel learning of the supervised/self-supervised/unsupervised learning mentioned above allows the numerical vector (embedding vector) output by the encoder to include clinical information shown in chest radiology and morphological information unrelated thereto simultaneously, thereby enhancing versatility (supervised/self-supervised learning), and efficiently relocates the vector space in which the numerical vector is placed (unsupervised training) to makes it possible to efficiently utilize the encoder for other types of downstream tasks that are not planned in advance. That is, this has an effect of further helping to implement few-shot, one-shot learning.

On the other hand, in the following embodiments, utilization examples of the encoder described above or numerical vectors extracted therefrom are as follows:

### Utilization Examples of Numerical Vectors

As a utilization example of the exemplary numerical vector of the present application, all additional information other than the numerical vector obtained from the encoder may be concatenated into one input vector and may be used to pass through a new network of the downstream task to perform desired clinical diagnosis, clinical event/treatment prediction.

The additional structured data information may include: at least one of existing structured information, such as a age, a gender or vital signs including blood pressure, pulse rate, body temperature, respiratory rate, oxygen saturation, and various laboratory test results; unstructured data converted into structured numerical information through a machine learning method (images, sounds, biosignals other than chest radiology); and natural language information, such as symptoms transformed into numerical vectors through natural language processing, diagnosis names, and medical records.

The network of the downstream task to be used may be a MLP neural network composed of two or more fully-connected layers including batch normalization, dropout layer, and non-linear activation function (e.g., Relu) already mentioned above as a preferred example, but the specific configuration may vary depending on the intended use.

When training a new network of the downstream task, as mentioned above, fine tuning may be applied to first fix the weights of the encoder, then update the weight of the new downstream network through training, and then update the entire weights of the encoder and the network of the downstream task through additional training.

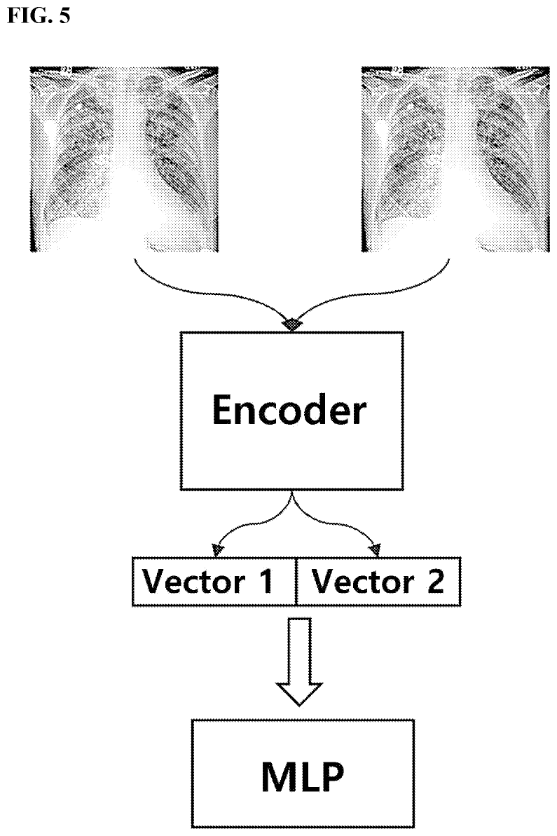

FIG. 5 is a diagram illustrating utilization of a numerical vector obtained from a plurality of pieces of chest radiology data obtained through repetitive measurement, according to another embodiment of the present application.

Referring to FIG. 5, chest radiology is often performed multiple times in one patient. When pneumonia or pulmonary edema is suspected, chest radiology is performed from hours to days. For patients in stable condition, chest radiology is performed at intervals of weeks to years. The evaluation through these repeated measurements refers to clinically evaluating the morphological changes of chest radiology by a physician over time to diagnose the risk of a particular disease/condition. In order to implement the same function through artificial intelligence, it is necessary to consistently quantify the atypical morphological features of each repeatedly implemented chest radiology data, and the encoder of the embodiment of the present application performs this role.

That is, first, by analyzing two chest radiology images that meet a specific clinical criterion (e.g., time interval), each chest radiology data is passed through a separate encoder (two ECG encoders may have the same parameter weights, i.e., parameter sharing), and two numerical vectors are concatenated to create one input numerical vector. Then, the network of the downstream task is generated in the manner mentioned above, wherein an input end is structured to be able to accept the input vector format, and an output end is structured to predict a specific diagnosis (or a group of diagnoses) to be predicted, so as to train the corresponding model. At this time, the time point of prediction/diagnosis is generally the time point of the most recently performed test. In this case, the utilization examples may include, but are not limited to, all types of pneumonia (and pulmonary infection), pulmonary edema, lung cancer, lung metastasis, (atrial and ventricular) cardiomegaly, change in cardiac function (left/right ventricular systolic function), cardiac valve stenosis/insufficiency, coronary artery calcium deposition and stenosis, interstitial lung disease, chronic obstructive pulmonary disease/pulmonary emphysema, improvement (shock amelioration) or worsening (heart failure/pulmonary edema) of the patient's condition before and after the fluid therapy, and the like.

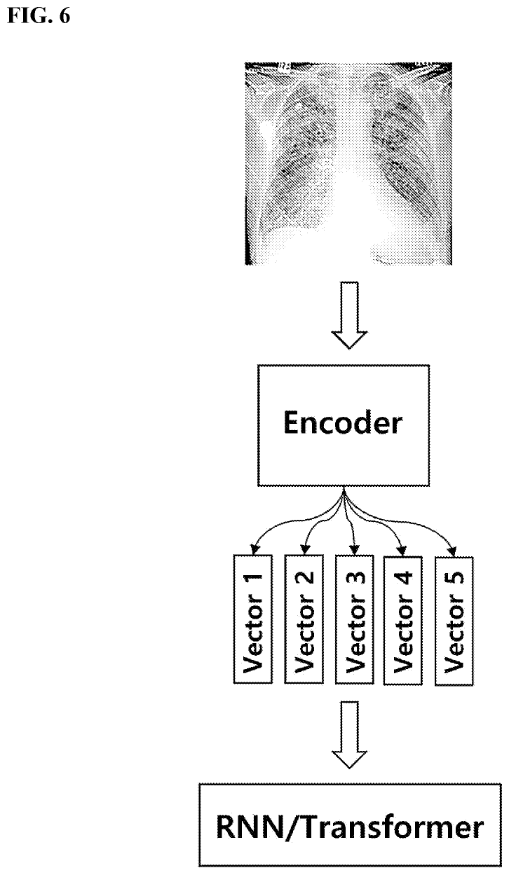

FIG. 6 is a diagram illustrating utilization of N sequentially obtained numerical vectors, according to another embodiment of the present application.

Referring to FIG. 6, N sequentially performed chest radiology data satisfying a specific clinical criterion are passed through one encoder 12. This corresponds to embedding of chest radiology data, which is unstructured data, through which N sequentially obtained numerical vectors are obtained. The sequential embedding vectors thus obtained can be input and passed through a general RNN (LSTM or GRU) or a transformer network to train and utilize a learning model that predicts whether a particular disease will improve/deteriorate or a particular clinical event will occur over time. Each of the sequential numerical vectors used as an input value may be supplemented by concatenating additional information, including clinical information (age, gender, blood pressure, pulse rate, respiratory rate, body temperature, symptoms, structured test results) converted into the numerical vectors. The RNN or the transformer network used herein is only one example of the neural network structure capable of processing numerical vectors sequentially configured through repeated measurements. Any other machine learning algorithm capable of performing a similar function may be used.

For example, the artificial intelligence algorithm for calculating or diagnosing the risk of pneumonia (and pulmonary infection), pulmonary edema, lung cancer, lung metastasis, (atrial and ventricular) cardiomegaly, changes in cardiac function (left/right ventricular systolic function), cardiac valve stenosis/insufficiency, coronary artery calcium deposition and stenosis, interstitial lung disease, chronic obstructive pulmonary disease/pulmonary emphysema, improvement (shock amelioration) or worsening (heart failure/pulmonary edema) of patient's condition before and after fluid therapy may be incorporated into a chest radiology machine, a PACS, or an EHR program by utilizing a plurality of chest radiology images that have been repeatedly measured.

The device for analyzing a disease described above may be implemented by a computing device that includes at least some of a processor, memory, a user input device, and a presentation device.

The memory is a medium that stores computer-readable software, applications, program modules, routines, instructions, and/or data, that are coded to be able to perform certain tasks when executed by the processor. The processor may read and execute computer-readable software, applications, program modules, routines, instructions, and/or data stored in the memory. The user input device may cause the user to input a command for causing the processor to execute a specific task or input data required for executing the specific task. The user input device may include a physical or virtual keyboard or keypad, a keybutton, a mouse, a joystick, a trackball, touch-sensitive input means, a microphone, or the like. The presentation device may include a display, a printer, a speaker, a vibrator, or the like.

The computing device may include a variety of devices, such as smartphones, tablets, laptops, desktops, servers, clients, and the like. The computing device may include a device having built-in functionality to analyze and output the chest radiology input or capable of communicating with external computing equipment that incorporates such functionality, such as a wearable device with a camera, e.g., camera-mounted glasses or a camera that can be attached to the body or clothes or integrated into an accessory. The computing device may be a single stand-alone device and may include multiple computing devices operating in a distributed environment of multiple computing devices cooperating with each other over a communication network.

The method of converting chest radiology data into numerical vectors described above may also be executed by the computing device having a processor and memory storing computer-readable software, applications, program modules, routines, instructions, and/or data structures coded to perform the method of converting chest radiology data into numerical vectors by converting the chest radiology data into numerical vectors while being executed by the processor.

The embodiments described above may be implemented through various means. For example, the present embodiments may be implemented by hardware, firmware, software, or a combination thereof.

For implementation by hardware, the method of converting chest radiology data into numerical vectors according to the present embodiments may be implemented by one or more application-specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, or the like.

For example, the method of converting chest radiology data into numerical vectors according to embodiments may be implemented using an artificial intelligence semiconductor device in which neurons and synapses of a deep neural network are implemented as semiconductor devices. In this case, the semiconductor device may be currently used semiconductor devices, such as SRAM, DRAM, NAND, or the like, or may be next-generation semiconductor devices, such as RRA, STT MRAM, PRAM, or a combination thereof.

When the method of analyzing a disease by converting chest radiology data into numerical vectors according to embodiments is implemented using the artificial intelligence semiconductor device, the result (weight) of learning the neural network model by software may be transferred to synapse-mimicking devices arranged in an array or learning may be performed in the artificial intelligence semiconductor device.

In the case of implementation by firmware or software, the method of analyzing a disease by converting chest radiology data into numerical vectors according to the present embodiments may be implemented in the form of a device, a procedure, or a function that performs the functions or operations described above. The software code may be stored in a memory unit and driven by a processor. The memory unit is located inside or outside the processor, so that data can be exchanged with the processor by various known means.

In addition, as described above, terms such as “unit”, “device”, “module”, “system”, “processor”, “controller”, “component”, “interface”, or “unit” may generally refer to computer-related entity hardware, a combination of hardware and software, software, or software in execution. For example, the foregoing components may be, but are not limited to, a process driven by a processor, a processor, a controller, a control processor, an entity, a thread of execution, a program, and/or a computer. For example, both an application running on the controller or processor, and the controller or the processor may be components. One or more components may be within a process and/or a thread of execution and may be located in one device (e.g., a system, and a computing device) or distributed across two or more devices.

The above description is merely an example of the technical idea of the present application. Those skilled in the art to which the present application pertains make various modifications and variations without departing from the essential features of the technical idea of the present application. In addition, the present embodiments are not intended to limit the technical idea of the present application, but are intended to describe the same. Therefore, the scope of the technical idea of the present application is not limited by the embodiments. The scope of protection of the present application should be interpreted by the following claims, and all technical ideas falling within the scope of equivalents thereof should be interpreted as being included in the scope of rights of the present application.

### INDUSTRIAL AVAILABILITY

According to exemplary implementations of the present application, it is possible to extract a structured numerical vector from atypical chest radiology, in particular chest radiology data, and to utilize the same in various clinical situations.

In particular, it is possible to extract general numerical information maximizing the utilization range of the chest radiology information while utilizing the existing clinical framework as it is. This general numerical information (embedding vector) is not only used as such, but also fused and utilized with other information of the patient. In addition, the change in the patient state can be easily quantified by quantifying the chest radiology data. Accordingly, the general numerical information can be usefully used for the initial evaluation and treatment response evaluation in a hospital room, an intensive care unit, and an emergency room. In addition, some of the structured numerical vectors are utilized as inputs to other artificial intelligence algorithms or clinical protocols for various diagnoses that can be associated with the chest radiology data.

What is claimed is

1. A device for analyzing a disease by converting chest radiology data into numerical vectors, the device comprising:
an acquisition unit for acquiring chest radiology data;
an encoder that receives the chest radiology data and uses a deep learning algorithm to calculate a first numerical vector; and
an analysis unit that uses the first numerical vector calculated by the encoder to provide an analysis result that is information regarding disease-related analysis, prediction, or diagnosis,
wherein the first numerical vector is structured data contextually including anatomical features that can be extracted from the chest radiology data, and being associated with features extracted from the chest radiology data.

2. The device of claim 1, further comprising one or more downstream task processing units that utilize the first numerical vector to process a downstream task, wherein error signals from each output end of a network of the downstream task are backpropagated to gather at the end of the encoder to train the encoder to improve versatility of the first numerical vector.

3. The device of claim 1, wherein the first numerical vector is used for machine learning.

4. The device of claim 1, wherein the information regarding disease-related diagnosis provided by the analysis unit includes rhythm abnormalities of the heart, including at least one or more of tachycardia, bradycardia, and various arrhythmias, and structure and function abnormalities of the heart, including at least one or more of heart failure, pericardial tamponade, valvular stenosis/failure, pulmonary hypertension, pulmonary embolism, and cardiomyopathy.

5. The device of claim 4, wherein the disease predicted and diagnosed by the analysis unit includes acute respiratory distress syndrome (ARDS), pneumonia, abscess, aspiration pneumonia, atypical pneumonia, active tuberculosis, non-tuberculous mycobacteria, chronic obstructive pulmonary disease (COPD), interstitial lung disease, bronchiectasis, sarcoidosis, lung nodule, lung mass, lung cancer, lung metastasis, aortic dissection, aortic aneurysm, pleural effusion, empyema, pneumothorax, pneumoperitoneum, pneumopericardium, pneumomediastinum, subcutaneous emphysema, coronary artery calcification, cardiomegaly, pulmonary edema, pericardial effusion, pulmonary embolism, chamber (LA, LV, RA, RV) enlargements, valvular (aortic, mitral, tricuspid, pulmonary) calcification/stenosis/regurgitation, hypertrophic cardiomyopathy, and various fractures, tumors, and metastasis of ribs, sternum, and spine.

6. The device of claim 2, wherein the one or more downstream task processing units further receive additional structured data input information including structured biometric information including an age, a gender, a blood pressure, a pulse rate, a respiratory rate, a body temperature, a laboratory test result, and unstructured information structured through modification including a chief complaint, an underlying disease, text, ultrasound image information, acoustic information, such as auscultation sound, and various biosignals, wherein the additional structured data input information is concatenated with the first numerical vector or input separately from the first numerical vector.

7. The device of claim 1, wherein the chest radiology data is a single-channel or multi-channel image, and the chest radiology data input to the encoder is in the form of a two-dimensional or three-dimensional array of C×W×H (the number of channels×the number of horizontal-axis pixels×the number of vertical-axis pixels).

8. The device of claim 1, wherein the chest radiology data is a chest radiology image, and the chest radiology image is resized and cropped to a particular size and normalized and input to the encoder.

9. A method of analyzing a disease by converting chest radiology data into numerical vectors, performed by a processor, the method comprising:
obtaining chest radiology data from a chest radiology measurement device;
inputting the chest radiology data into an encoder;
calculating a first numerical vector by using deep learning through the encoder; and
performing a disease-related analysis, prediction, or diagnosis using the first numerical vector.

10. The method of claim 9, further comprising processing one or more downstream tasks by utilizing the first numerical vector, wherein error signals from each output end of a network of the downstream task are backpropagated to gather at the end of the encoder to train the encoder to improve versatility of the first numerical vector.

11. A computer-readable recording medium for storing program instructions readable by a computer and operable by the computer, wherein the program instructions, when executed by a processor of the computer, cause the processor to perform the method of claim 9.
